SEQUENCE LISTING (29 sequences)

SEQ ID NO: 1 - protein, 745 aa, Homo sapiens
MAQQTSPDTLTVPEVDNPHCPNPWLNEDLVKSLRENLLQHEKSKTARKSVSPKLSPVISPRNSPRLLRRMLLSSNIPKQRRFTVAHTCFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 2 - protein, 5 aa, synthetic construct
PVISP

SEQ ID NO: 3 - protein, 45 aa, synthetic construct
  VARIANT 1: note = may be absent or N
  VARIANT 2: note = may be absent or L
  VARIANT 3: note = may be absent or L
  VARIANT 4: note = may be absent or Q
  VARIANT 5: note = may be absent or H
  VARIANT 6: note = may be absent or E
  VARIANT 7: note = may be absent or K
  VARIANT 8: note = may be absent or S
  VARIANT 9: note = may be absent or K
  VARIANT 10: note = may be absent or T
  VARIANT 11: note = may be absent or A
  VARIANT 12: note = may be absent or R
  VARIANT 13: note = may be absent or K
  VARIANT 14: note = may be absent or S
  VARIANT 15: note = may be absent or V
  VARIANT 16: note = may be absent or S
  VARIANT 17: note = may be absent or P
  VARIANT 18: note = may be absent or K
  VARIANT 19: note = may be absent or L
  VARIANT 20: note = may be absent or S
  VARIANT 26: note = may be absent or R
  VARIANT 27: note = may be absent or N
  VARIANT 28: note = may be absent or S
  VARIANT 29: note = may be absent or P
  VARIANT 30: note = may be absent or R
  VARIANT 31: note = may be absent or L
  VARIANT 32: note = may be absent or L
  VARIANT 33: note = may be absent or R
  VARIANT 34: note = may be absent or R
  VARIANT 35: note = may be absent or M
  VARIANT 36: note = may be absent or L
  VARIANT 37: note = may be absent or L
  VARIANT 38: note = may be absent or S
  VARIANT 39: note = may be absent or S
  VARIANT 40: note = may be absent or N
  VARIANT 41: note = may be absent or I
  VARIANT 42: note = may be absent or P
  VARIANT 43: note = may be absent or K
  VARIANT 44: note = may be absent or Q
  VARIANT 45: note = may be absent or R
XXXXXXXXXXXXXXXXXXXXPVISPXXXXXXXXXXXXXXXXXXXX

SEQ ID NO: 4 - protein, 25 aa, synthetic construct
NLLQHEKSKTARKSVSPKLSPVISP

SEQ ID NO: 5 - protein, 25 aa, synthetic construct
EKSKTARKSVSPKLSPVISPRNSPR

SEQ ID NO: 6 - protein, 25 aa, synthetic construct
ARKSVSPKLSPVISPRNSPRLLRRM

SEQ ID NO: 7 - protein, 25 aa, synthetic construct
SPKLSPVISPRNSPRLLRRMLLSSN

SEQ ID NO: 8 - protein, 25 aa, synthetic construct
PVISPRNSPRLLRRMLLSSNIPKQR

SEQ ID NO: 9 - protein, 5 aa, synthetic construct
KSLRE

SEQ ID NO: 10 - protein, 45 aa, synthetic construct
  VARIANT 1: note = may be absent or T
  VARIANT 2: note = may be absent or V
  VARIANT 3: note = may be absent or P
  VARIANT 4: note = may be absent or E
  VARIANT 5: note = may be absent or V
  VARIANT 6: note = may be absent or D
  VARIANT 7: note = may be absent or N
  VARIANT 8: note = may be absent or P
  VARIANT 9: note = may be absent or H
  VARIANT 10: note = may be absent or C
  VARIANT 11: note = may be absent or P
  VARIANT 12: note = may be absent or N
  VARIANT 13: note = may be absent or P
  VARIANT 14: note = may be absent or W
  VARIANT 15: note = may be absent or L
  VARIANT 16: note = may be absent or N
  VARIANT 17: note = may be absent or E
  VARIANT 18: note = may be absent or D
  VARIANT 19: note = may be absent or L
  VARIANT 20: note = may be absent or V
  VARIANT 26: note = may be absent or N
  VARIANT 27: note = may be absent or L
  VARIANT 28: note = may be absent or L
  VARIANT 29: note = may be absent or W
  VARIANT 30: note = may be absent or H
  VARIANT 31: note = may be absent or E
  VARIANT 32: note = may be absent or K
  VARIANT 33: note = may be absent or S
  VARIANT 34: note = may be absent or K
  VARIANT 35: note = may be absent or T
  VARIANT 36: note = may be absent or A
  VARIANT 37: note = may be absent or R
  VARIANT 38: note = may be absent or K
  VARIANT 39: note = may be absent or S
  VARIANT 40: note = may be absent or V
  VARIANT 41: note = may be absent or S
  VARIANT 42: note = may be absent or P
  VARIANT 43: note = may be absent or K
  VARIANT 44: note = may be absent or L
  VARIANT 45: note = may be absent or S
XXXXXXXXXXXXXXXXXXXXKSLREXXXXXXXXXXXXXXXXXXXX

SEQ ID NO: 11 - protein, 25 aa, synthetic construct
TVPEVDNPHCPNPWLNEDLVKSLRE

SEQ ID NO: 12 - protein, 25 aa, synthetic construct
DNPHCPNPWLNEDLVKSLREMLLQH

SEQ ID NO: 13 - protein, 25 aa, synthetic construct
PNPWLNEDLVKSLRENLLQHEKSKT

SEQ ID NO: 14 - protein, 25 aa, synthetic construct
NEDLVKSLRENLLQHEKSKTARKSV

SEQ ID NO: 15 - protein, 25 aa, synthetic construct
KSLRENLLWHEKSKTARKSVSPKLS

SEQ ID NO: 16 - protein, 20 aa, synthetic construct
SPDTLTVPEVDNPHCPNPWL

SEQ ID NO: 17 - protein, 30 aa, synthetic construct
  VARIANT 1: note = may be absent or M
  VARIANT 2: note = may be absent or A
  VARIANT 3: note = may be absent or Q
  VARIANT 4: note = may be absent or Q
  VARIANT 5: note = may be absent or T
  VARIANT 26: note = may be absent or N
  VARIANT 27: note = may be absent or E
  VARIANT 28: note = may be absent or D
  VARIANT 29: note = may be absent or L
  VARIANT 30: note = may be absent or V
XXXXXSPDTLTVPEVDNPHCPNPWLXXXXX

SEQ ID NO: 18 - protein, 25 aa, synthetic construct
MAQQTSPDTLTVPEVDNPHCPNPWL

SEQ ID NO: 19 - protein, 25 aa, synthetic construct
SPDTLTVPEVDNPHCPNPWLNEDLV

SEQ ID NO: 20 - protein, 25 aa, synthetic construct
TVPEVDNPHCPNPWLNEDLVKSLRE

SEQ ID NO: 21 - protein, 25 aa, synthetic construct
DNPHCPNPWLNEDLVKSLRENLLQH

SEQ ID NO: 22 - protein, 25 aa, synthetic construct
PNPWLNEDLVKSLRENLLQHEKSKT

SEQ ID NO: 23 - protein, 25 aa, synthetic construct
NEDLVKSLRENLLQHEKSKTARKSV

SEQ ID NO: 24 - protein, 25 aa, synthetic construct
KSLRENLLQHEKSKTARKSVSPKLS

SEQ ID NO: 25 - protein, 25 aa, synthetic construct
NLLQHEKSKTARKSVSPKLSPVISP

SEQ ID NO: 26 - protein, 25 aa, synthetic construct
EKSKTARKSVSPKLSPVISPRNSPR

SEQ ID NO: 27 - protein, 25 aa, synthetic construct
ARKSVSPKLSPVISPRNSPRLLRRM

SEQ ID NO: 28 - protein, 25 aa, synthetic construct
SPKLSPVISPRNSPRLLRRMLLSSN

SEQ ID NO: 29 - protein, 25 aa, synthetic construct
PVISPRNSPRLLRRMLLSSNIPKRQ

(19) United States
(12) Patent Application Publication
(10) Pub. No.: US 2024/0325504 A1
(43) Pub. Date: Oct. 3, 2024

(54) TREATMENT

(71) Applicants: Glasgow Caledonian University, Glasgow (GB); University Court of the University of Glasgow, Glasgow (GB)

(72) Inventors: Niall Macquaide, Glasgow (GB); George Baillie, Glasgow (GB)

(21) Appl. No.: 18/619,958

(22) Filed: Mar. 28, 2024

(30) Foreign Application Priority Data
Mar. 31, 2023 (GB) 2304853.1

(51) Int. Cl. A61K 38/46; A61K 38/08

(52) U.S. Cl. CPC A61K 38/465; A61K 38/08

(57) ABSTRACT

Described herein are molecules and compounds for use in medicine, as medicaments and for the treatment or prevention of cardiac diseases.

A Sequence Listing in XML text format, entitled 9013.226_ST26.xml, 60,751 bytes in size, generated on Mar. 28, 2024, and filed herewith, is hereby incorporated by reference into the specification for its disclosures.

### FIELD

Disclosed are molecules and compounds for use in medicine, as medicaments and for the treatment or prevention of cardiac diseases.

### BACKGROUND

Phosphodiesterases (PDE) regulate local cellular cAMP levels and are targeted to many cellular proteins which are PKA substrates. Phospholamban (PLB) phosphorylation is known to be regulated by PDE3A, and is also antiarrhythmic¹. Phosphorylation of PLB by PKA relieves PLB-mediated inhibition of SERCA2a. Both PLB phosphorylation and SERCA2a expression/activity are downregulated in heart failure².

There is a need for therapeutic options that can be used to improve contraction in heart failure (HF) patients.

### SUMMARY

The present disclosure is based on the finding that PDE4D5 associates with the SERCA complex to regulate phospholamban (PLB) phosphorylation.

Sarco/endoplasmic reticulum Ca²⁺-ATPase (SERCA) activity is inhibited by PLB. Increased CAMP activates protein kinase A (PKA), phosphorylating PLB relieving SERCA inhibition, increasing Ca2+ uptake and contraction.

Phospholamban phosphorylation is well established, regulated by PDE3A and antiarrhythmic. Without wishing to be bound by theory, the phosphorylation of PLB by PKA relieves PLB-mediated inhibition of SERCA2a. Both PLB phosphorylation and SERCA2a expression/activity are downregulated in heart failure.

An association between PDE4D5 and the SERCA complex controls local cyclic 3′, 5′-adenosine monophosphate (cAMP) levels and regulates and the phosphorylation of PLB (by PKA). Modulating the interaction between PDE4D5 and SERCA results in a local increase in CAMP which in turn leads to increased PLB phosphorylation and improves contraction in heart failure patients.

In view of the above, this disclosure provides a molecule (for example a peptide) which modulates the interaction between phosphodiesterase 4D (PDE4D), phospholamban (PLB) and/or sarcoplasmic reticulum Ca²⁺-ATPase (SERCA) for use in medicine or as a medicament.

The disclosure also provides a molecule (for example a peptide) which modulates the interaction between phosphodiesterase 4D (PDE4D), phospholamban (PLB) and/or sarcoplasmic reticulum Ca2+-ATPase (SERCA) for use in the treatment of a cardiac disease.

This disclosure provides molecules (for example peptides) for use as inotropic therapies.

The disclosure also provides molecules (for example peptides) for use in the treatment or prevention of myocardial infarction (MI).

A molecule (for example a peptide) of this disclosure may interfere with and/or inhibit interactions between phosphodiesterase 4D (PDE4D), phospholamban (PLB) and/or sarcoplasmic reticulum Ca2+-ATPase (SERCA).

A molecule (for example a peptide) of this disclosure may promote the uptake of Ca²⁺ by SERCA from the cytosol of the cardiomyocyte and/or reduce PDE4D-dependent hydrolysis of CAMP near the SERCA complex, increasing the phosphorylation of phospholamban (PLB).

In one teaching, the molecule may comprise or consist essentially of or consist of a peptide.

A peptide of this disclosure may be derived from the PDE4D5 sequence. An exemplary (human) PDE4D5 sequence may comprise the sequence given as SEQ ID NO: 1 below.

SEQ ID NO: 1.
maqqtspdtl tvpevdnphc pnpwlnedlv kslrenllqh
eksktarksv spklspvisp rnsprllrrm llssnipkqr
rftvahtcfd vingtsagrs pldpmtspgs glilqanfvh
sqrresflyr sdsdydlspk smsrnssias dihgddlivt
pfaqvlaslr tvrnnfaalt nlqdrapskr spmenqpsin
katiteeayq klasetleel dwcldqletl qtrhsvsema
snkfkrmlnr elthlsemsr sgnqvsefis ntfldkqhev
eipsptqkek ekkkrpmsqi sgvkklmhss sltnssiprf
gvkteqedvl akeledvnkw glhvfriael sgnrpltvim
htifqerdll ktfkipvdtl itylmtledh yhadvayhnn
ihaadvvqst hvllstpale avftdleila aifasaihdv
dhpgvsnqfl intnselalm yndssvlenh hlavgfkllq
eencdifqnl tkkqrqslrk mvidivlatd mskhmnllad
lktmvetkkv tssgvllldn ysdriqvlqn mvhcadlsnp
tkplqlyrqw tdrimeeffr qgdrerergm eispmcdkhn
asveksqvgf idyivhplwe twadlvhpda qdildtledn
rewyqstipq spspapddpe egrqgqtekf qfeltleedg
esdtekdsgs qveedtscsd sktlctqdse steipldeqv
eeeavgeeee sqpeacvidd rspdt

A peptide of this disclosure may comprise SEQ ID NO: 1 or a fragment or portion thereof. Useful fragments of SEQ ID NO: 1 may be functional in that they exhibit the same or substantially the same activity as the native full peptide. For example, where the full peptide prevents binding between PDE4D5 and any constituent of the SERCA complex, any fragment or portion of that full peptide may also exhibit that same property. As such, functional fragments of SEQ ID NO: 1 may modulate, for example inhibit, interactions and/or binding between PDE4D5 and SERCA. Useful fragments of SEQ ID NO: 1 may comprise anywhere between about 5 amino acids and n-1 amino acids, where n=745 (i.e. the total number of residues making up SE ID NO: 1).

A peptide of this disclosure may comprise the amino acid sequence of SEQ ID NO: 1

SEQ ID NO: 2
PVISP

A peptide of this disclosure may comprise SEQ ID NO: 2 and an additional 1-20, for example 2, 3, 4, 5, 6, 7, 8, 9, 10, 11, 12, 13, 14, 15, 16, 17, 18 or 19 amino acids. The additional amino acids may be added to the N and/or C-terminus of the peptide provided by SEQ ID NO: 2.

A useful peptide may comprise the following sequence:

SEQ ID NO: 3
X₁X₂X₃X₄X₅X₆X₇X₈X₉X₁₀X₁₁X₁₂X₁₃X₁₄X₁₅X₁₆X₁₇X₁₈X₁₉X₂₀PVISPX₂₁X₂₂
X₂₃X₂₄X₂₅X₂₆X₂₇X₂₈X₂₉X₃₀X₃₁X₃₂X₃₃X₃₄X₃₅X₃₆X₃₇X₃₈X₃₉X₄₀

Wherein X₁-X₄₀ are each independently an amino acid or absent.
x₁ may be absent or N
x₂ may be absent or L
x₃ may be absent or L
x₄ may be absent or Q
x₅ may be absent or H
x₆ may be absent or E
x₇ may be absent or K
x₈ may be absent or S
x₉ may be absent or K
x₁₀ may be absent or T
x₁₁ may be absent or A
x₁₂ may be absent or R
x₁₃ may be absent or K
x₁₄ may be absent or S
x₁₅ may be absent or V
x₁₆ may be absent or S
x₁₇ may be absent or P
x₁₈ may be absent or K
x₁₉ may be absent or L
x₂₀ may be absent or S
x₂₁ may be absent or R
x₂₂ may be absent or N
x₂₃ may be absent or S
x₂₄ may be absent or P
x₂₅ may be absent or R
x₂₆ may be absent or L
x₂₇ may be absent or L
x₂₈ may be absent or R
x₂₉ may be absent or R
x₃₀ may be absent or M
x₃₁ may be absent or L
x₃₂ may be absent or L
x₃₃ may be absent or S
x₃₄ may be absent or S
x₃₅ may be absent or N
x₃₆ may be absent or I
x₃₇ may be absent or P
x₃₈ may be absent or K
x₃₉ may be absent or Q
x₄₀ may be absent or R

A peptide of this disclosure may comprise, consist essentially of or consist of any of the following sequences, or a functional fragment thereof:

SEQ ID NO: 4
NLLQHEKSKTARKSVSPKLSPVISP
SEQ ID NO: 5
EKSKTARKSVSPKLSPVISPRNSPR
SEQ ID NO: 6
ARKSVSPKLSPVISPRNSPRLLRRM
SEQ ID NO: 7
SPKLSPVISPRNSPRLLRRMLLSSN
SEQ ID NO: 8
PVISPRNSPRLLRRMLLSSNIPKQR

A peptide of this disclosure may comprise the amino acid sequence of SEQ ID NO: 9

SEQ ID NO: 9
KSLRE

A peptide of this disclosure may comprise SEQ ID NO: 9 and an additional 1-20, for example 2, 3, 4, 5, 6, 7, 8, 9, 10, 11, 12, 13, 14, 15, 16, 17, 18 or 19 amino acids. The additional amino acids may be added to the N and/or C-terminus of the peptide provided by SEQ ID 9.

A useful peptide may comprise the following sequence:

SEQ ID NO: 10
X₄₁X₄₂X₄₃X₄₄X₄₅X₄₆X₄₇X₄₈X₄₉X₅₀X₅₁X₅₂X₅₃X₅₄X₅₅X₅₆X₅₇X₅₈X₅₉X₆₀KSL
REX₆₁X₆₂X₆₃X₆₄X₆₅X₆₆X₆₇X₆₈X₆₉X₇₀X₇₁X₇₂X₇₃X₇₄X₇₅X₇₆X₇₇X₇₈X₇₉X₈₀

Wherein X₄₁-X₈₀ are each independently an amino acid or absent.

By way of example:
x₄₁ may be absent or T
x₄₂ may be absent or V
x₄₃ may be absent or P
x₄₄ may be absent or E
x₄₅ may be absent or V
x₄₆ may be absent or D
x₄₇ may be absent or N
x₄₈ may be absent or P
x₄₉ may be absent or H
x₅₀ may be absent or C
x₅₁ may be absent or P
x₅₂ may be absent or N
x₅₃ may be absent or P
x₅₄ may be absent or W
x₅₅ may be absent or L
x₅₆ may be absent or N
x₅₇ may be absent or E
x₅₈ may be absent or D
x₅₉ may be absent or L
x₆₀ may be absent or V
x₆₁ may be absent or N
x₆₂ may be absent or L
x₆₃ may be absent or L
x₆₄ may be absent or W
x₆₅ may be absent or H
x₆₆ may be absent or E
x₆₇ may be absent or K
x₆₈ may be absent or S
x₆₉ may be absent or K
x₇₀ may be absent or T
x₇₁ may be absent or A
x₇₂ may be absent or R
x₇₃ may be absent or K
x₇₄ may be absent or S
x₇₅ may be absent or V
x₇₆ may be absent or S
x₇₇ may be absent or P
x₇₈ may be absent or K
x₇₉ may be absent or L
x₈₀ may be absent or S

A useful peptide may comprise, consist essentially of or consist of any of the following sequences, or a functional fragment thereof:

SEQ ID NO: 11
TVPEVDNPHCPNPWLNEDLVKSLRE
SEQ ID NO: 12
DNPHCPNPWLNEDLVKSLREMLLQH
SEQ ID NO: 13
PNPWLNEDLVKSLRENLLQHEKSKT
SEQ ID NO: 14
NEDLVKSLRENLLQHEKSKTARKSV
SEQ ID NO: 15
KSLRENLLWHEKSKTARKSVSPKLS

A useful peptide may comprise the amino acid sequence of SEQ ID NO: 16

SEQ ID NO: 16
SPDTLTVPEVDNPHCPNPWL

A peptide of this disclosure may comprise SEQ ID NO: 16 and an additional 1-10, for example 2, 3, 4, 5, 6, 7, 8, 9, or 10 amino acids. The additional amino acids may be added to the N and/or C-terminus of the peptide provided by SEQ ID 16.

A peptide of this disclosure may comprise the following sequence:

SEQ ID NO: 17
X₈₁X₈₂X₈₃X₈₄X₈₅SPDTLTVPEVDNPHCPNPWLX₈₆X₈₇X₈₈X₈₉X₉₀

Wherein x₈₁-x₉₀ are each independently an amino acid or absent.

By way of example:
x₈₁ may be absent or M
x₈₂ may be absent or A
x₈₃ may be absent or Q
x₈₄ may be absent or Q
x₈₅ may be absent or T
x₈₆ may be absent or N
x₈₇ may be absent or E
x₈₈ may be absent or D
x₈₉ may be absent or L
x₉₀ may be absent or V

A peptide of this disclosure may comprise, consist essentially of or consist of any of the following sequences, or a functional fragment thereof:

SEQ ID NO: 18
MAQQTSPDTLTVPEVDNPHCPNPWL
SEQ ID NO: 19
SPDTLTVPEVDNPHCPNPWLNEDLV

The term functional fragment may embrace any fragment or portion of any of the peptides described herein, which fragment or portion exhibits the same or substantially the same activity as the native full peptide. For example, where the full peptide prevents binding between PDE4D5 and any constituent of the SERCA complex, any fragment or portion of that full peptide may also exhibit that same property. As such, functional fragments of this disclosure may modulate, for example inhibit, interactions and/or binding between PDE4D5 and SERCA.

A functional fragment may comprise 5 or more amino acids from any of the peptides described herein. For example, a functional fragment may comprise 6, 7, 8, 9, 10, 11, 12, 13, 14, 15, 16, 17, 18, 19, 20, 21, 22, 23 or 24 (consecutive) amino acids of any of the peptides disclosed herein.

The disclosure may also embrace peptides which are functional variants or derivatives of any of the peptides described herein (including those provided by SEQ ID NOS: 1-19).

A functional variant or derivative may modulate (for example inhibit or prevent) binding between PDE4D5 and SERCA. A functional variant may comprise, relative to a reference sequence, one or more amino acid modifications. The term ‘amino acid’ modifications may comprise amino acid substitutions, additions and/or deletions. The term ‘amino acid’ modifications may comprise amino acid substitutions, additions and/or deletions. The term ‘amino acid’ modifications may comprise one or more conservative substitutions, where one or more of the (wild type) amino acids of any of the peptides of this disclosure are substituted for a different amino acid with similar biochemical properties (e.g. charge, hydrophobicity and size) or an amino acid separated from the wild type amino acid by a small physicochemical distance. Accepted conservative substitutions are well known to one of skill.

A functional variant or derivative may comprise, an amino acid sequence which shares a degree of identity or homology a reference sequence. For example, a functional variant or derivative may comprise a sequence which is at least 50%, 55%, 60%, 65%, 70%, 75%, 80%, 85%, 90%, 91%, 92%, 93%, 94%, 95%, 96%, 97%, 98% or 99% identical or homologous to all or part (e.g. a functional part) of a reference sequence.

It should be noted that the term ‘reference sequence’ may embrace any of the peptide sequences described herein, including any peptide comprising, consisting essentially of or consisting of, a sequence of SEQ ID NOS: 1-19.

In view of the above, the present disclosure provides any one of SEQ ID NOS: 1-19 or functional fragments, variants or derivatives of any of these:
(i) for use in medicine; and/or
(ii) for use as a medicament; and/or
(iii) for use in treating or preventing a cardiac disease; and/or
(iv) for use in treating heart failure; and/or
(v) for use in improving contraction of the heart; and/or
(vi) for use as an inotropic therapy; and/or
(vii) for use in the treatment or prevention of myocardial infarction (MI).

The disclosure further provides compositions comprising any of the peptides disclosed herein.

A composition of this disclosure may be a pharmaceutical composition.

A composition of this invention may be sterile and may further comprise one or more (pharmaceutically acceptable) excipients, diluents and/or carriers.

A method of identifying test agents which modulate binding between PDE4D5 and SERCA, said method comprising contacting a test agent with PDE4D5 and SERCA and determining whether or not the test agent modulates biding between PDE4D5 and SERCA.

The test agent may be a peptide. The test agent may be derived from the full length PDE4D5 sequence.

The step of determining whether or not the test agent modulates biding between PDE4D5 and SERCA, may comprise detecting complexes comprising PDE4D5 and proteins that form the SERCA complex (e.g. PLB, AKAP18δ or AKAP18γ). Such complexes may be referred to as PDE4D5/SERCA complexes It should be noted that the term PDE4D5/SERCA complex means a complex in which PDE4D5 is bound to SERCA and/or a protein which form part of the SERCA complex. In a method of this disclosure, detection of a PDE4D5/SERCA complex indicates that the test agent has not modulated (for example interfered with or prevented) biding between PDE4D5 and SERCA. Where the method fails to report the presence of PDE4D5/SERCA complexes, this may indicate that the test agent may have modulated (for example interfered with, inhibited or prevented) binding between PDE4D5 and SERCA.

Test agents which modulate (e.g. inhibit, prevent or interfere with) binding between PDE4D5 and SERCA may be:
(i) for use in medicine; and/or
(ii) for use as a medicament; and/or
(iii) for use in treating or preventing a cardiac disease; and/or
(iv) for use in treating heart failure; and/or
(v) for use in improving contraction of the heart; and/or
(vi) for use as an inotropic therapy; and/or
(vii) for use in the treatment or prevention of myocardial infarction (MI). The disclosure also provides a method of treating or preventing:

The disclosure also provides methods of treating or preventing:
(i) a cardiac disease; and/or
(ii) heart failure; and/or
(iii) myocardial infarction (MI);
   or
(iv) improving contraction of the heart; and/or
(v) delivering an inotropic therapy;
said method comprising administering a subject in need thereof (a therapeutically effective) amount of:
(i) a molecule (for example a peptide) which modulates the interaction between phosphodiesterase 4D (PDE4D), phospholamban (PLB) and/or sarcoplasmic reticulum Ca2+-ATPase (SERCA); and/or
(ii) a molecule (for example a peptide) promoting the uptake of Ca2+ by SERCA from the cytosol of the cardiomyocyte and/or diminishing PDE4D-dependent de-phosphorylation of phospholamban (PLB); and/or
(iii) a peptide of this disclosure; and/or
(iv) a peptide comprising any of the sequences provided by SEQ ID NO: 1-8 or a (functional) fragment thereof.

The term ‘subject’ may embrace human or animal subjects suffering from cardiac disease, heart failure or myocardial infarction. The term subject may further include human or animal subjects convalescing from cardiac disease, heart failure or myocardial infarction or human or animal subjects predisposed or susceptible to cardiac disease, heart failure or myocardial infarction.

The disclosure also provides the use of:
(i) a molecule (for example a peptide) which modulates the interaction between phosphodiesterase 4D (PDE4D), phospholamban (PLB) and/or sarcoplasmic reticulum Ca2+-ATPase (SERCA); and/or
(ii) a molecule (for example a peptide) promoting the uptake of Ca2+ by SERCA from the cytosol of the cardiomyocyte and/or diminishing PDE4D-dependent de-phosphorylation of phospholamban (PLB); and/or
(iii) a peptide of this disclosure; and/or
(iv) a peptide comprising any of the sequences provided by SEQ ID NO: 1-8 or a (functional) fragment thereof;

In the manufacture of a medicament for treating or preventing:
(i) a cardiac disease; and/or
(ii) heart failure; and/or
(iii) myocardial infarction (MI);
or
(iv) improving contraction of the heart; and/or
(v) delivering an inotropic therapy.

### DETAILED DESCRIPTION

The disclosure will now be described by reference to the following Figures:

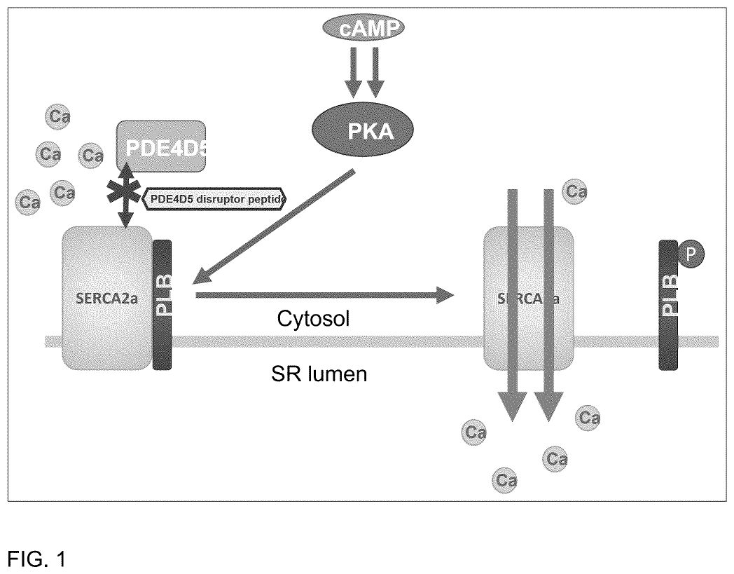

FIG. 1. Regulation of SERCA activity by cAMP. SERCA activity is inhibited by PLB at rest. Increased cAMP activates PKA, phosphorylating PLB relieving SERCA inhibition, increasing Ca2+ uptake and contraction. A PDE associated with SERCA controls CAMP levels, regulating PKA phosphorylation of PLB. Preventing this PDE from binding to SERCA should allow a local increase in cAMP, resulting in increased PLB phosphorylation.

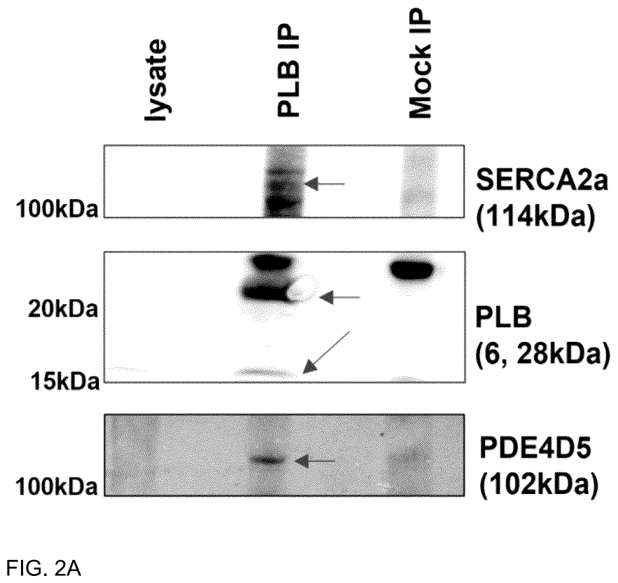

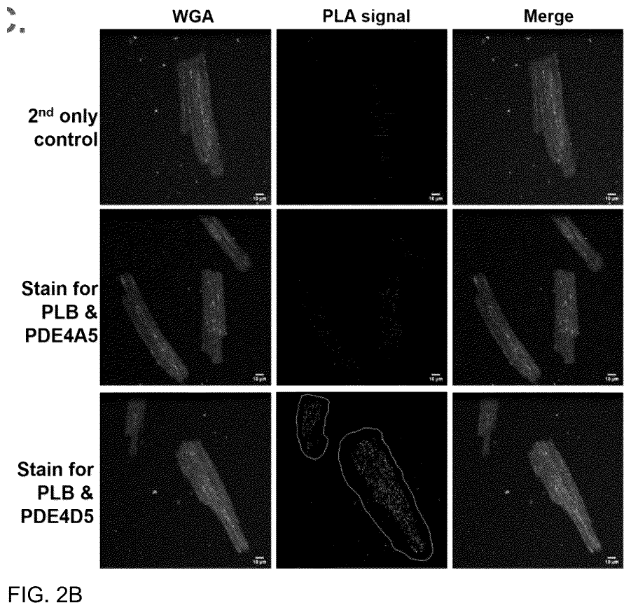

FIGS. 2A-2B. PDE4D5 is expressed in adult rabbit cardiomyocytes and associated with SERCA2a/PLB complex. (FIG. 2A). co-immunoprecipitation and (FIG. 2B). proximity ligation assay (PLA) (N=3). Arrows in (FIG. 2A) indicates the correct bands in each blot.

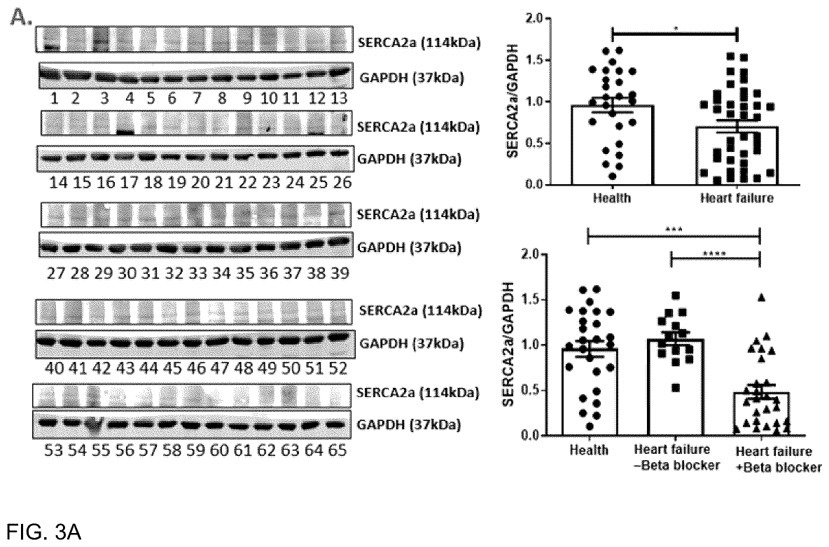

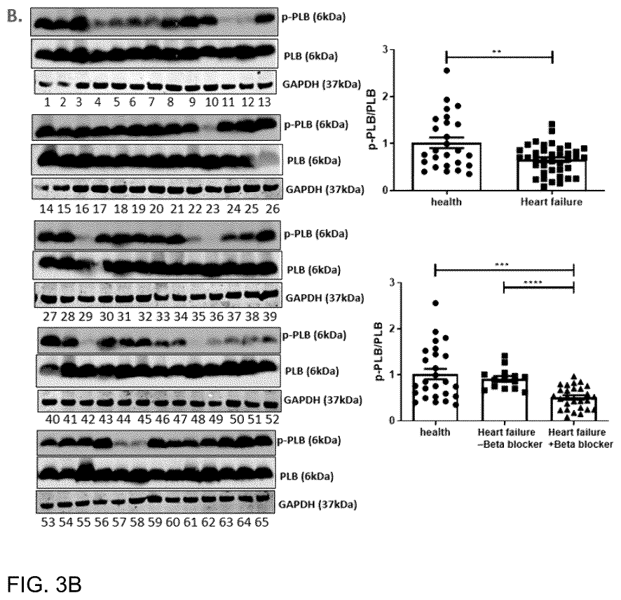

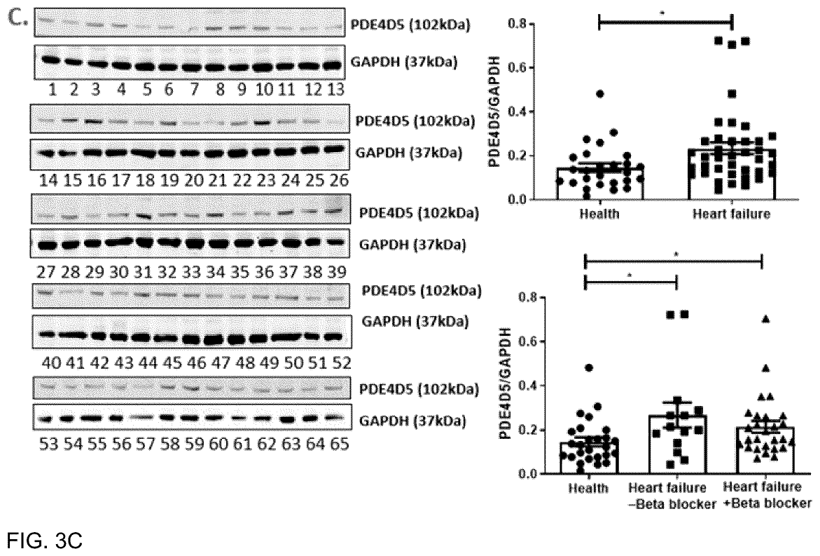

FIGS. 3A-3C. Tissue samples from 65 human hearts were collected from heart failure patient and healthy donors. All western blots were performed double blinded. Western blotting in each panel show alternate control and HF samples across each blot. All the samples were further catalogued into healthy group, heart failure patients that has been treated with beta-blocker before heart transplant surgery or not. (FIG. 3A). SERCA2a expression decreased in heart failure human heart tissue, similar to Lipskaia et al³. Patients that have been treated with beta-blocker have substantially reduced in SERCA2a expression compared with non-beta-blocker or healthy patients. (p<0.05, N=26, 39) (FIG. 3B). PLB phosphorylation is decreased in HF samples. (p<0.01, N=26, 39). Patients with beta-blocker group has the lowest PLB phosphorylation level compared to others. (FIG. 3C). PDE4D5 expression increased in HF patients. This agrees with increased PDE4 in HF patients observed by Richter et al⁴. (p<0.05, N=26, 39).

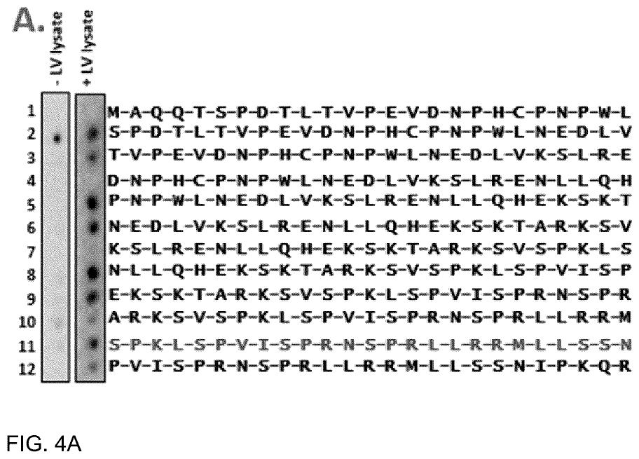

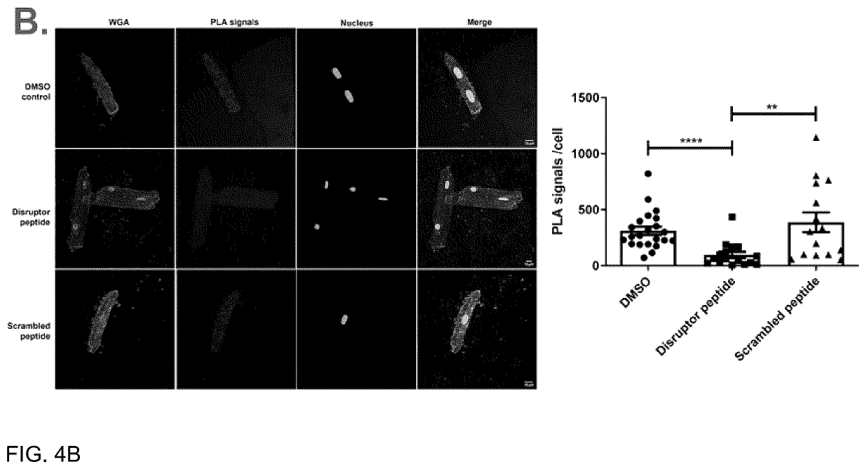

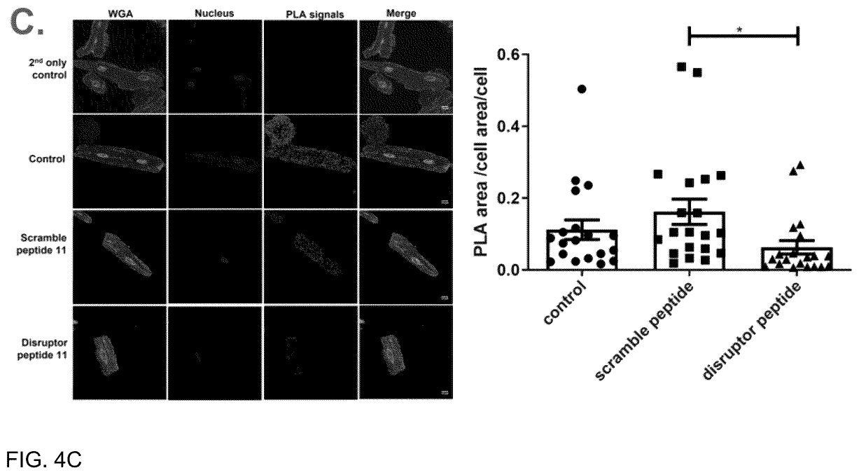

FIGS. 4A-4C. PDE4D5-SERCA complex disruptor peptide successfully prevents PDE4D5 binding. (FIG. 4A). Peptide array mapping on PDE4D5 sequences to detect interaction site for PDE4D5-SERCA/PLB interaction. Full length PDE4D5 sequence were created utilizing 25-mer overlapping peptides with a 5-amino acid shift between spots on membranes. Membranes were incubated with adult rabbit heart lysates then incubated with SERCA2a antibody to investigate the interaction motif. Black dot represents binding signal. Sequence 11 was chosen as a disruptor peptide for further studies. (FIG. 4B). Interaction between PLB and PDE4D5 was decreased after disruptor peptide treatment. detected by PLA. (N=3, **p<0.01, ****p<0.0001). (FIG. 4C) Interaction between SERCA2a and PDE4D5 were decreased after disruptor peptide treatment (N=3, **p<0.01, ****p<0.0001).

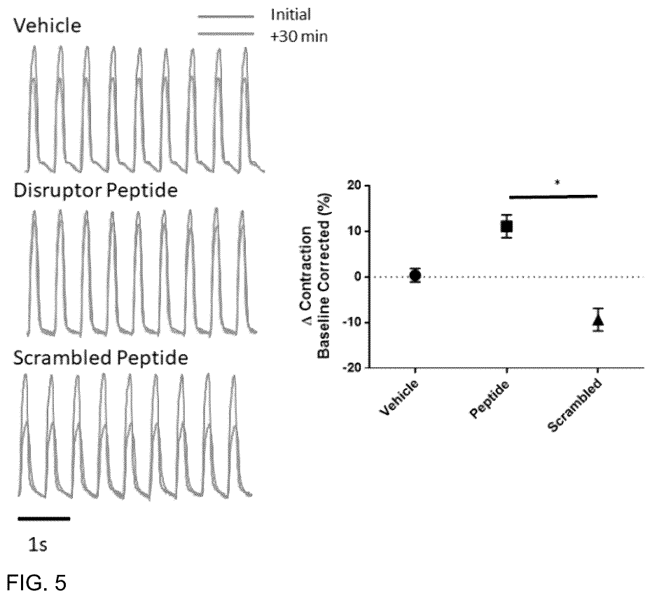

FIG. 5. Disruptor peptide increases contractility in MI rabbit ventricular myocytes. (Left) Contraction measurements in 3 example myocytes after 30 minutes incubation with vehicle, disruptor peptide and scrambled peptide. (Right) the graph shows preliminary mean+/−SEM data from 3 MI rabbits (p<0.05, biological repeats N=3, technical repeats n=45).

PDE4D5 is the only PDE4 isoform that associates with the PLB/SERCA complex as determined using (A). mass spectrometry techniques (see Tables 1 and 2)

TABLE 1
XP 008257794.1 Mass 124434 Score: 437 Matches: 10(10 Sequences 9(9) emPAI: 0.31
Predicted: cGMP-inhibited 3′,5′-cyclic phosphodiesterase
A isoform X1 [Oryctolagus cuniculus]

| Query | Observed | Mr(expt) | Mr(calc) | ppm | Miss | Score | Expect | Rank | Unique | Peptide |
|---|---|---|---|---|---|---|---|---|---|---|
| 2542 | 483.2744 | 964.5343 | 964.5342 | 0.18 | 0 | 52 | 0.0001 | 1 | U | R.ILSQVSRY.L |
| 2955 | 503.2827 | 1004.5508 | 1004.5502 | 0.6 | 0 | 33 | 0.012 | 1 | U | R.TVSLTSLER.F |
| 5716 | 736.3994 | 1470.7843 | 1470.7831 | 0.83 | 0 | 76 | 9.7E−07 | 1 | U | R.YVEQILPQSAAPR.E |
| 6387 | 777.8820 | 1553.7494 | 1553.7474 | 1.26 | 0 | 42 | 0.003 | 1 | U | R.SFSSSYAVSAANHVK.V |
| 6702 | 796.8994 | 1591.7843 | 1591.7842 | 0.05 | 0 | 48 | 0.00096 | 1 | U | K.ISTVQFPESADTAAR.Q |
| 7625 | 888.3939 | 1774.7732 | 1774.7758 | −1.49 | 0 | 99 | 4.8E−09 | 1 | U | K.VNDEVGIDWTNENDR.L |
| 7663 | 595.6389 | 1783.8949 | 1783.8951 | −0.12 | 1 | (40) | 0.0051 | 1 | U | K.IQAIEEEEEEKGKPR.A |
| 7664 | 892.9563 | 1783.8980 | 1783.8951 | 1.63 | 1 | 95 | 1.5E−08 | 1 | U | K.IQAIEEEEEEKGKPR.A |
| 8750 | 1026.5300 | 2051.0454 | 2051.0323 | 6.39 | 0 | 68 | 6.7E−06 | 1 | U | R.TNAFLVATSAPQAVLYNDR.S |
| 9213 | 1101.5300 | 2201.0454 | 2201.0335 | 5.42 | 0 | 75 | 2E−06 | 1 | U | R.LTGIENQSVEQTPPQQSSEK.I |

TABLE 2
XP 008260467.1 Mass: 64851 Score: 34 Matches 2(2) Sequences 1(1) emPAI: 0.05
PREDICTED: CAMP-specific 3′5-cyclo phosphodiesterase
4D isoform X2 [Oryctolagus cuniculus]

| Query | Observed | Mr(expt] | Mr(cale) | ppm | Miss | Score | Expect | Rank | Unique | Peptide |
|---|---|---|---|---|---|---|---|---|---|---|
| 1427 | 434.7668 | 867.5190 | 867.5178 | 1.41 | 0 | 29 | 0.014 | 1 | 1 | K.LSPVISPR.N |

### CONCLUSIONS

PDE4D5 is responsible for regulating SERCA activity via modulation of PLB phosphorylation.

Targeted disruption of PDE4D5-SERCA complex interaction is a promising inotropic therapy in MI.

The present invention will now be described with reference to the following numbered clauses:

Clause 1. A molecule which modulates the interaction between phosphodiesterase 4D (PDE4D), phospholamban (PLB) and/or sarcoplasmic reticulum Ca2+-ATPase (SERCA) for use in medicine or as a medicament.

Clause 2. A molecule which modulates the interaction between phosphodiesterase 4D (PDE4D), phospholamban (PLB) and/or sarcoplasmic reticulum Ca2+-ATPase (SERCA) for use in the treatment or prevention of (i) a cardiac disease, (ii) myocardial infarction (MI).

Clause 3. The molecule of clause 1 or 2, for use of clause 1 or 2, wherein the molecule is a peptide.

Clause 4. The molecule of any preceding clause, for use of any preceding clause, wherein the molecule comprises a sequence provided by any of SEQ ID NOS 1-19 or a functional fragment thereof.

Clause 5. The molecule of any preceding claim, for use of any preceding claim, wherein the molecule comprises any one of SEQ ID NOS: 4-8, 11-16 or 18-19.

Clause 6. A composition comprising a molecule comprising a peptide according to any one of SEQ ID NOS: 1-19 or a functional fragment thereof.

Clause 7. The composition of clause 6, wherein the composition is a pharmaceutical composition.

Clause 8. A method of identifying test agents which modulate binding between PDE4D5 and SERCA, said method comprising contacting a test agent with PDE4D5 and SERCA and determining whether or not the test agent modulates biding between PDE4D5 and SERCA.

Clause 9. The method of clause 8, wherein the test agent is derived from SEQ ID NO: 1.

### REFERENCES

Ahmad, F., Shen, W., Vandeput, F., Szabo-Fresnais, N., Krall, J., Degerman, E., Goetz, F., Klussmann, E., Movsesian, M., & Manganiello, V. (2015). Regulation of Sarcoplasmic Reticulum Ca 2 ATPase 2 (SERCA2) Activity by Phosphodiesterase 3A (PDE3A) in Human Myocardium PHOSPHORYLATION-DEPENDENT INTERACTION OF PDE3A1 WITH SERCA2*. Journal of Biological Chemistry, 290, 6763-6776. https://doi.org/10.1074/jbc.M115.638585
Ablorh, N.-A. D., & Thomas, D. D. (2015). Phospholamban phosphorylation, mutation, and structural dynamics: a biophysical approach to understanding and treating cardiomyopathy. https://doi.org/10.1007/s12551-014-0157-z
Lipskaia, L., Chemaly, E. R., Hadri, L., Lompre, A.-M., & Hajjar, R. J. (2010). Sarcoplasmic reticulum Ca 2+ ATPase as a therapeutic target for heart failure. https://doi.org/10.1517/14712590903321462
Richter, W., Xie, M., Scheitrum, C., Krall, J., Movsesian, M. A., & Conti, M. (2011). Conserved expression and functions of PDE4 in rodent and human heart. https://doi.org/10.1007/s00395-010-0138-8

What is claimed is:

1. A method of treating or preventing a disease or condition, said method comprising administering to a subject in need thereof a molecule which modulates the interaction between phosphodiesterase 4D (PDE4D), phospholamban (PLB) and/or sarcoplasmic reticulum Ca2+-ATPase (SERCA).

2. The method of claim 1, wherein the method is for the treatment or prevention of (i) a cardiac disease or (ii) myocardial infarction (MI).

3. The method of claim 1, wherein the method is for the treatment or prevention of
(i) treating heart failure; and/or
(ii) improving contraction of the heart.

4. The method of claim 1, wherein the molecule promotes the uptake of Ca2+ by SERCA from the cytosol of the cardiomyocyte and/or reduce PDE4D-dependent hydrolysis of CAMP near the SERCA complex, increasing the phosphorylation of phospholamban (PLB).

5. The method of claim 1, wherein the molecule is a peptide.

6. The method of claim 1, wherein the molecule comprises a sequence provided by any of SEQ ID NOS 1-19 or a functional fragment thereof.

7. The method of claim 1, wherein the molecule comprises any one of SEQ ID NOS: 4-8, 11-16 or 18-19.

8. A composition comprising a molecule comprising a peptide according to any one of SEQ ID NOS: 1-19 or a functional fragment thereof.

9. The composition of claim 8, wherein the composition is a pharmaceutical composition.

10. A method of identifying test agents which modulate binding between PDE4D5 and SERCA, said method comprising contacting a test agent with PDE4D5 and SERCA and determining whether or not the test agent modulates biding between PDE4D5 and SERCA.

11. The method of claim 10, wherein the test agent is derived from SEQ ID NO: 1.
